(19) United States
(12) Patent Application Publication
(10) Pub. No.: US 2006/0068046 A1
(43) Pub. Date: Mar. 30, 2006

(54) Zinc (ii)-rich natural materials using plant

(75) Inventors: Junichi Arita, Takamatsu (JP); Yoshitane Kojima, Izumi (JP); Yutaka Yoshikawa, Toyonaka (JP); Munekazu Gemba, Yawata (JP); Yoshiko Kawai, Matsubara (JP); Yuka Koda, Hirakata (JP)

Correspondence Address: Koda & Androlia, Suite 1140, 2029 Century Park East, Los Angeles, CA, 90067-3024, US

(73) Assignee: Junicihi Arita, Kagawa (JP)

(21) Appl. No.: 10/537,706

(22) PCT Filed: Nov. 25, 2003
(86) PCT No.: PCT/JP03/15051

§ 371 Date: Jun. 3, 2005

(30) Foreign Application Priority Data
Dec. 5, 2002 (JP) 2002-353886
May 15, 2003 (JP) 2003-136659

(51) Int. Cl. A61K 36/185

(52) U.S. Cl. 424/777

(57) ABSTRACT

To relieve symptoms caused by mineral deficiency such as taste abnormality and skin damage due to zinc deficiency, anemia due to iron deficiency and decreased bone mineral content due to calcium deficiency to thereby improve quality of life (QOL). Namely, foods such as foods for specific health uses and foods with nutrient function claims containing 0.1% or marc, based on gram of a dry natural plant material comprising a papaya powder etc., of zinc which are prepared by nonproliferatively stirring and/or shaking the natural plant material in a state of being suspended in a solution containing 100 ppm or greater of zinc.

### TECHNICAL FIELD

The present invention relates to natural food substances containing minerals, especially zinc, in high concentrations, and a method for manufacturing the same.

### BACKGROUND ART

Zn-containing natural substances contained in plants and foodstuffs generally have a low content of minerals such as zinc, etc. As a result, numerous disorders caused by zinc deficiency, such as abnormalities in the sense of taste, etc., are generated. Similarly, calcium, magnesium and/or iron and the like also play important roles in the body and are minerals that are commonly deficient. Foodstuffs that allow the sufficient ingestion of such minerals are not to be found. Mineral yeast-Zn has been reported as a conventional technique (for example, see Patent Reference 1).

The following may be cited as prior art information relating to the invention of the present application.

Patent Reference 1: Japanese Patent Application Laid-Open (Kokai) No. H08-32082 (pp. 2-7)

In the case of an ordinary diet, it is extremely difficult to ensure ingestion of the necessary amount of zinc and/or other minerals, and to maintain an appropriate ingestion balance. In the living body, zinc is contained in approximately 300 types of enzymes and proteins, and plays an important role in active sites and the like. In recent years, mineral deficiencies have become a serious problem as a result of abnormalities in the sense of taste and skin disorders caused by zinc deficiency, anemia caused by iron deficiency, a drop in the amount of bone salts caused by calcium deficiency and the like.

For example, in the case of the amount of zinc contained in natural substances such as papaya, maca and the like, this amount is insufficient from the standpoint of supply to the body. In order to overcome this problem, it is necessary to provide natural substances which have a lower toxicity than zinc (II) ions and which have a good stability, good fat-solubility and high zinc content, as food products.

### DISCLOSURE OF INVENTION

The present invention relates to food products containing zinc sources and natural substances that contain zinc in high concentrations.

For example, papaya, maca and the like may be cited as examples of natural substances that may contain zinc and that may be used in the present invention. However, the present invention is not limited to these substances.

The zinc sources used in the present invention may be any zinc sources that are suitable for administration to humans or other animals. For instance, mineral salts of zinc, organic zinc complexes and the like may be cited as desirable examples.

Examples of mineral salts of zinc include zinc acetate, zinc sulfate, zinc chloride, zinc nitrate and the like. Furthermore, in cases where mineral salts of zinc are used as zinc sources, for example, basic aqueous solutions of potassium hydroxide, sodium hydroxide, lithium hydroxide, barium hydroxide or the like, or buffer solutions such as citric acid buffers, phosphoric acid buffers or the like, may be used in combination with these salts as pH adjusting agents.

For example, organic zinc complexes having as ligands compounds selected from the group consisting of amino acids, picolinic acids, niacins, vitamins, maltols, carboxylic acids, oligopeptides, sugars and organic substances (mainly natural substances) having derivatives of these compounds may be cited as desirable examples of organic zinc complexes.

Furthermore, the food products of the present invention containing zinc in high concentrations may also contain other foods, food additives, vitamins and minerals.

In regard to these other foods, food additives, minerals and the like, such products may be products that are ordinarily used in the field of foods and the like, or products that may be developed in the future.

The form of the food products used in the present invention may be powder form, granular, tablet form, capsule form liquid form, gel form or any other form.

### BRIEF DESCRIPTION OF DRAWINGS

FIG. 1 shows the relationship of the zinc concentration in liquid to the zinc concentration taken into powdered dry papaya.

### BEST MODE FOR CARRYING OUT THE INVENTION

The following embodiments are shown to illustrate the present invention, and it should be noted that the present invention is not limited to the described embodiments.

### EMBODIMENT 1

Using zinc sulfate as a zinc source, solutions were prepared so that the zinc ion concentrations of these solutions ranged from 100 to 100,000 ppm. In the zinc solutions of respective concentrations, powdered papaya was suspended at the rate of 10 g per 100 ml, and these suspensions were shaken for four (4) days at room temperature (amplitude 12 cm, 100 rpm).

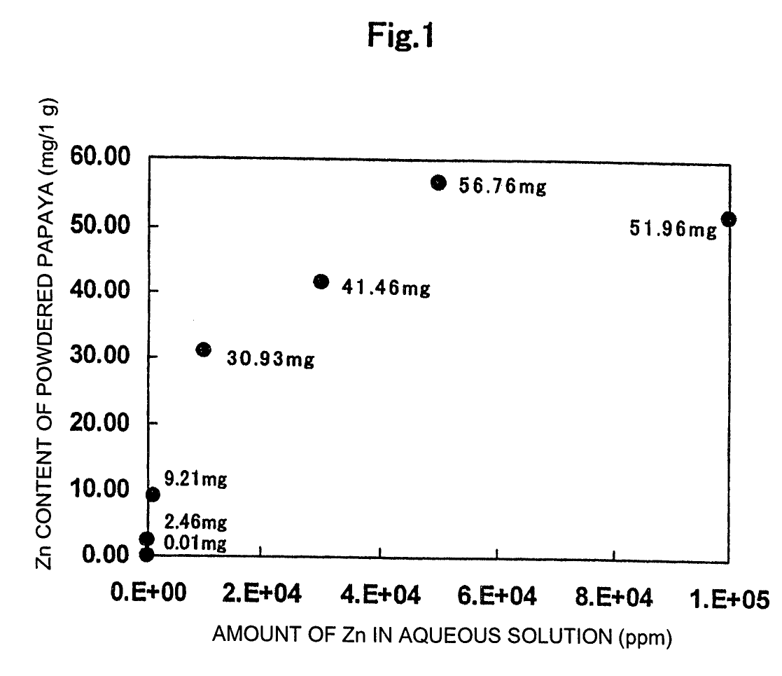

Following shaking, the powdered papaya was separated by centrifuging, and washing was performed three times using water purified by a reverse osmosis membrane. Afterward, the powdered papaya as dried under reduced pressure, and the zinc was determined using an atomic absorption mass spectrometer (FIG. 1).

Results and Discussion

The powdered papaya showed an increase in zinc content depending on the concentration of the solution, and more or less reached saturation at a concentration of 30,000 ppm or greater.

### INDUSTRIAL APPLICABILITY

The substances with a high zinc content provided by the present invention ameliorate impairment of the sense of taste caused by zinc deficiency and improve the state of health of patients suffering from hyperlipemia, diabetes and the like, which are diseases arising from lifestyle habits. These substances of the present invention are also useful in the treatment and prevention of atherosclerosis, cardiovascular disease, hyperglycemia, angina, hypertension, congestive heart failure, complications of diabetes and the like.

Furthermore, a diet effect as a result of ingestion as ordinary foodstuffs may also be expected. In addition, the food products of the present invention show no substantial side effects even in the case of long-term ingestion and are gentle and safe for humans.

1. A natural food product that contains plants (papaya, maca or the like) which contain zinc in high concentrations.

2. A natural food product that contains zinc at a rate of 1,000 ppm or greater, preferably 10,000 ppm or greater.

3. A method for manufacturing the natural food product (papaya, maca or the like) which contains zinc in large amounts according to claim 1 or 2, characterized in that plants are stirred and/or shaken in a non-proliferating manner in a suspended state under aerobic conditions in a solution that contains a mineral salt or organic complex of zinc as a zinc source at a rate of 100 ppm or greater.

4. The food product according to claim 1 or 2, characterized in that said food product uses a natural substance that contains minerals in high concentrations as said natural substance.

5. A food product that contains calcium, magnesium and/or iron in large amounts according to claim 1 or 2, characterized in that mineral is calcium, magnesium and/or iron.

6. A method for manufacturing a natural food product that contains calcium, magnesium and/or iron in large amounts according to claim 1 or 2, characterized in that a natural substance is stirred and/or shaken in a non-proliferating manner in a suspended state under aerobic conditions in a solution that contains a mineral salt or organic complex as a source of calcium, magnesium and/or iron at a rate of 100 ppm or greater.
